(19) United States
(12) Patent Application Publication
(10) Pub. No.: US 2025/0009415 A1
(43) Pub. Date: Jan. 9, 2025

(54) SURGICAL SYSTEM WITH LOCATION DETECTION

(71) Applicant: Boston Scientific Medical Device Limited, Galway (IE)

(72) Inventors: Gareth Davies, Toronto (CA); Kaylie Lau, Toronto (CA)

(21) Appl. No.: 18/763,055

(22) Filed: Jul. 3, 2024

Related U.S. Application Data
(60) Provisional application No. 63/512,413, filed on Jul. 7, 2023.

(51) Int. Cl. A61B 18/14; A61B 18/00

(52) U.S. Cl. CPC A61B 18/1477; A61B 2018/00363; A61B 2018/00839

(57) ABSTRACT

A system to access an epicardium of a heart is disclosed. The system includes a stimulation probe and a controller. The stimulation probe is advanced toward a pericardium and provides an electrical stimulation signal of a selected amplitude and frequency. The controller is coupled to the to the stimulation probe. The controller provides the electrical stimulation signal of the selected amplitude and frequency to the stimulation probe and adjusts the selected amplitude, frequency, or both of the electrical stimulation signal based on a detected stimulation of the heart in response to the electrical stimulation signal.

### CROSS REFERENCE TO RELATED APPLICATION

The present application claims priority to U.S. Provisional Patent Application No. 63/512,413, filed Jul. 7, 2023, the entire disclosure of which is incorporated herewith in its entirety.

### TECHNICAL FIELD

The present disclosure relates to medical devices, systems and methods for use in surgical procedures. More specifically, this disclosure relates to surgical devices, systems and methods for cutting and access of bodily tissues and providing information regarding proximity to the heart.

### BACKGROUND

The epicardium, or outer layer of the heart, is accessed for several cardiac procedures. Traditionally, the epicardium or epicardial space has been accessed for such procedures as catheter ablation of ventricular arrhythmias and accessory pathway. The epicardium or epicardial space is also accessed to treat left atrial appendage occlusion, esophageal protection, mapping and ablation during atrial fibrillation procedures, implantation of epicardial pacing leads, and phrenic nerve displacement to facilitate safe ablation of atrial and ventricular arrhythmias. Accessing the epicardial space, however, is a challenge and typically involves intimate knowledge of cardiac anatomy, extensive training, and expertise to avoid issues such as inadvertent right ventricular perforation. Multiple technological advances have led to significant improvements in epicardial access success. Examples of advances include carbon dioxide insufflation through the coronary sinus or the right atrial appendage, pressure sensors on needles, computed tomography, cardiac magnetic resonance, and electroanatomic mapping guided access.

### SUMMARY

In Example 1, a system to access an epicardium of a heart, the system comprising: a stimulation probe configured to advance toward a pericardium and configured to provide an electrical stimulation signal of a selected amplitude and frequency; and a controller coupled to the to the stimulation probe, the controller configured to provide the electrical stimulation signal of the selected amplitude and frequency to the stimulation probe and adjust at least one of the selected amplitude and frequency of the electrical stimulation signal based on a detected stimulation of the heart in response to the electrical stimulation signal.

In Example 2, the system of Example 1, wherein the adjusting at least one of the selected amplitude and frequency includes lowering the amplitude.

In Example 3, the system of any of Examples 1-2, wherein the stimulation probe includes a needle configured to pierce the pericardium.

In Example 4, the system of Example 3, wherein the needle is a Tuohy needle.

In Example 5, the system of Example 3, wherein the needle is included in a micropuncture needle set.

In Example 6, the system of any of Examples 1-2, wherein the stimulation probe includes a delivery sheath.

In Example 7, the system of Example 6, wherein the delivery sheath includes an electrode.

In Example 8, the system of any of Examples 1-7, wherein the stimulation probe includes an electrode configured in a monopolar mode.

In Example 9, the system of any of Examples 1, 2 and 8, wherein the stimulation probe is a radio-frequency (RF) crossing device.

In Example 10, the system of any of Examples 1-9, wherein the controller is configured to detect the stimulation of the heart in response to the electrical stimulation signal.

In Example 11, the system of any of Examples 1-10, wherein the detected stimulation of the heart is based on a QRS wave of the heart.

In Example 12, the system of any of Examples 1-10, wherein the detected stimulation of the heart is based on changes in an electrogram signal.

In Example 13, the system of any of Examples 1-10, wherein the detected stimulation of the heart is based on fluoroscopy.

In Example 14, the system of any of Examples 1-12, wherein the controller is configured to determine whether the stimulation probe is touching the pericardium.

In Example 15, the system of any of Examples 1-14, wherein the controller is configured to provide a visualization.

In Example 16, a method of epicardial access of a heart, the method comprising: providing a puncture device including a shaft having a proximal portion and a distal portion, the distal portion including an electrode; providing a first stimulation signal having a first amplitude and frequency having a first stimulation energy to the electrode; advancing the electrode of the puncture device toward a pericardium of the heart by manipulating the proximal portion of the puncture device; detecting a first stimulation of the heart in response to the first stimulation signal; providing a second stimulation signal having a second stimulation signal having a second amplitude and frequency having a second stimulation energy to the electrode; and detecting a second stimulation of the heart in response to the second stimulation signal, based on the electrode contacting the epicardium of the heart; wherein the first stimulation energy is greater than the second stimulation energy.

In Example 17, the method of Example 16, wherein upon detecting a stimulation of the heart in response to the electrical stimulation signal, determining whether the stimulation probe is touching the pericardium.

In Example 18, the method of Example 17, wherein if the stimulation probe is touching the pericardium, piercing the pericardium.

In Example 19, the method of Example 17, wherein if the stimulation probe is not touching the pericardium, further advancing the stimulation probe toward the pericardium, the stimulation probe providing the adjusted at least one of the amplitude and frequency of the electrical stimulation signal.

In Example 20, the method of Example 16, wherein the adjusting at least one of the selected amplitude and frequency includes lowering the amplitude.

In Example 21, the method of Example 16, wherein the stimulation probe includes a needle configured to pierce the pericardium.

In Example 22, the method of Example 21, wherein the needle is a Tuohy needle.

In Example 23, the method of Example 21, wherein the needle is included in a micropuncture needle set.

In Example 24, the method of Example 16, wherein the stimulation probe includes a delivery sheath.

In Example 25, the method of Example 24, wherein the delivery sheath includes an electrode.

In Example 26, the method of Example 16, wherein the stimulation probe includes an electrode configured in a monopolar mode.

In Example 27, the method of Example 16, wherein the detecting the stimulation includes detecting the stimulation via a QRS wave of the heart.

In Example 28, the method of Example 16, wherein the detecting the stimulation includes detecting the stimulation via determining changes in an electrogram signal.

In Example 29, the method of Example 16, wherein the puncture device is a radio-frequency (RF) crossing device.

In Example 30, a system to access an epicardium of a heart, the system comprising: a stimulation probe configured to advance toward a pericardium and configured to provide an electrical stimulation signal of a selected amplitude and frequency; and a controller coupled to the to the stimulation probe, the controller configured to provide the electrical stimulation signal of the selected amplitude and frequency to the stimulation probe and adjust at least one of the selected amplitude and frequency of the electrical stimulation signal based on a detected stimulation of the heart in response to the electrical stimulation signal.

In Example 31, the system of Example 30, wherein the adjusting at least one of the selected amplitude and frequency includes lowering the amplitude.

In Example 32, a system to access an epicardium of a heart, the system comprising: a puncture device including a shaft having a proximal portion and a distal portion, the distal portion including an electrode, the puncture device configured to advance toward a pericardium of the heart by manipulating the proximal portion of the puncture device; and a controller coupled to the puncture device, the controller configured to provide a first stimulation signal having a first amplitude and frequency having a first stimulation energy to the electrode, detect a first stimulation of the heart in response to the first stimulation signal, provide a second stimulation signal having a second stimulation signal having a second amplitude and frequency having a second stimulation energy to the electrode, and detect a second stimulation of the heart in response to the second stimulation signal, based on the electrode contacting the epicardium of the heart; wherein the first stimulation energy is greater than the second stimulation energy.

In Example 33, the system of Example 32, wherein the puncture device is one of a radio-frequency (RF) crossing device and a needle configured to pierce the pericardium.

In Example 34, the system of Example 33, wherein the needle is one of a Tuohy needle and a micropuncture needle set.

In Example 35, the system of Example 32, wherein the controller is further configured to generate a visualization on a display.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, the example clinical setting having an example system including an example diagnostic controller in combination with a stimulation probe.

FIG. 2A is a schematic diagram illustrating a first example stimulation probe for use with the example system of FIG. 1.

FIG. 2B is a schematic diagram illustrating a second example stimulation probe for use with the example system of FIG. 1

FIG. 3 is a flow chart illustrating a method of the system of FIG. 1.

FIG. 4 is a schematic diagram illustrating the example system of FIG. 1 to implement the example method of FIG. 3.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

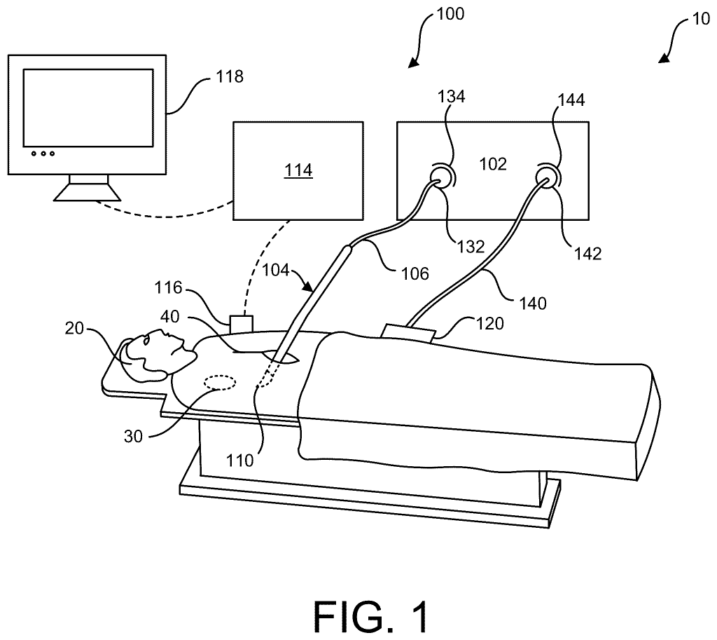

FIG. 1 illustrates an example of a clinical setting 10 for treating a patient 20, such as a heart 30 of the patient 20. In one example, the clinical setting is configured to access an epicardium of the heart of the patient, such as to perform one or more of several procedures such as catheter ablation of ventricular arrhythmias and accessory pathway. Clinical setting 10 includes a system 100 having a diagnostic controller 102 in combination with a stimulation probe 104. The diagnostic controller 102, in one example, is configured to generate an adjustable electrical stimulation signal of a selected amplitude and frequency. The stimulation probe 104 is electrically coupled to the diagnostic controller 102 and configured to receive the electrical stimulation signal, such as via a cable 106, and apply the electrical stimulation signal to the patient 20, such as via an electrode 110 disposed on the stimulation probe 104 such as on a distal end region of the stimulation probe. The stimulation probe 104 is also configured to enter the patient 20, such as via a skin incision 40, and be advanced through tissue toward the pericardium of the patient 20. In one embodiment, the stimulation probe 104 is configured with an electrode 110 and also configured to cut or puncture tissue of the patient such as the pericardium.

Epicardial access is achieved via puncturing a layer of pericardium while avoiding a myocardium of the heart. The pericardium is a tough, double-walled, fibroelastic sac encompassing the heart and the roots of the great vessels. The pericardium includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium and over the heart. This creates a pouch-like virtual or potential space around the heart enclosed between the two opposing serosal surfaces, often referred to as the pericardial space or pericardial cavity. The pericardial cavity is filled with a small amount of serous fluid, and defines a middle mediastinum. During ventricular contraction, a wave of depolarization moves from the endocardium of the heart to the epicardial surface of the heart. The pericardium serves to separate the heart from interference of other structures, and the serous fluid protects the heart against infection and blunt trauma, and lubricates the movements of the heart.

The system 100 further includes a recording system 112 including a controller 114 operably coupled to the patient 20 via a patient interface 116. The recording controller 114, in the example, is configured to monitor activity of the heart 30 and to detect a stimulation of the heart 30 in response to the electrical stimulation signal provided via the stimulation probe 104. In the illustrated recording system 112 of system 100, the recording controller 114 is operably coupled to a graphical display 118 to provide a visualization of heart activity and the stimulation of the heart 30 in response to the electrical stimulation signal. In one embodiment, the recording controller 114 is an electrocardiogram (ECG) recorder that is operably coupled to the patient via ECG electrode as the patient interface 116. The ECG recorder can monitor and generate a visualization on display 118 of the patient's heart rhythm or electrical activity. A stimulation of the heart 30 in response to the stimulation signal can be detected in the electrically activity, such as in the QRS wave, which represents ventricular depolarization, as a change to the normal heart rhythm or electrical activity in response to the presence of the electrical stimulation signal. Electrogram signals acquired by the patient interface 116 can be amplified, clipped, filtered, and otherwise processed with the recording controller 114 prior to being presented in a visualization to display 118.

In another embodiment of recording system 112, the recording controller 114 is an echocardiogram recorder that is operably coupled to the patient via an ultrasonic transducer as the patient interface 116. The echocardiogram recorder can monitor and generate a visualization of display 118 of the patient's heart movements or activity. A stimulation of the heart 30 in response to the stimulation signal can be detected in the heart movements or activity as a change to the normal heart movements or activity in response to the presence of the electrical stimulation signal.

In still another embodiment of recording system 112, the recording controller 114 is a fluoroscopy recorder that is operably coupled to the patient via a C-Arm or live X-ray device as the patient interface 116. The fluoroscopy recorder can monitor and generate a visualization of display 118 of the patient's heart movements or activity. A stimulation of the heart 30 in response to the stimulation signal can be detected in the heart movements or activity as a change to the normal heart movements or activity in response to the presence of the electrical stimulation signal.

The diagnostic controller 102 can provide monopolar, or both monopolar and bipolar stimulation signal output to a specified instrument such as stimulation probe 104 or a plurality or suite of instruments applied in the clinical setting 10. In one example, the diagnostic controller 102 is capable of simultaneously powering specified monopolar and bipolar instruments but may include a lock out feature preventing both monopolar and bipolar output from being simultaneously activated.

During monopolar operation of stimulation probe 104, a first electrode, often referred to as the active electrode 110, is provided with the stimulation probe 104 while a second electrode 120, often referred to as the indifferent or neutral electrode, is provided in the form of a ground pad dispersive electrode located on the patient 20. In one embodiment, the active electrode 110 is a conductive component on an instrument or the stimulation probe 104, such as a ring conductor, blade, or needle attached to a distal tip of the stimulation probe or a plurality of conductors attached to the stimulation probe, configured to receive the electrical stimulation signal. In another embodiment, an entire conductive instrument is configured to receive the stimulation signal. The ground pad dispersive electrode 120 is typically disposed on the back, buttocks, upper leg, or other suitable anatomical location during a procedure. In such a configuration, the ground pad dispersive electrode 120 is often referred to as a patient return electrode. In this configuration, an electrical circuit of stimulation energy is formed between the active electrode 110 and the ground pad dispersive electrode 120 through the patient 20 via the stimulation signal provided to the stimulation probe 104.

As the stimulation probe 104 is advanced toward the pericardium, the stimulation signal grows in strength with respect to the heart in the electrical circuit within the patient 20 and stimulates the heart. The stimulation of the heart in response to the electrical signal is detected via the recording system 112. The stimulation signal can be adjusted, such as reducing one or both of the power and frequency of the stimulation signal, and the stimulation probe 104 is further advanced toward the pericardium. As the stimulation signal again grows in strength with respect to the heart in the electrical circuit within the patient 20 as the stimulation probe is further advanced toward the pericardium, the stimulation signal will stimulate the heart again. The stimulation of the heart in response to the electrical signal is detected via the recording system 112. The stimulation signal can be adjusted, such as reducing one or both of the power and frequency of the stimulation signal. This process can continue to be repeated until the probe 104 is determined to be touching the pericardium. Once the probe 104 is determined to be touching the pericardium, the probe 104 can be applied to pierce the outer layer of the pericardium to access the epicardial space.

The cable 106 is included to mechanically and electrical couple the stimulation probe 104 to the diagnostic controller 102. Cable 106 includes plug 132 that connect with a receptacle 134 on the diagnostic controller 102. In one embodiment, the cable 106 is a single pin cable that is configured to receive and provide stimulation energy from the diagnostic controller 102 to the stimulation probe 104. In another embodiment, the cable 106 can include a plurality of conductors and associated plugs, and a receptacle can correspond with an active electrode receptacle and one or more receptacles can correspond with controls on the stimulation probe 104. In one embodiment, both ends of the cable 106 include a releasable locking mechanism to secure a mechanical and electrical connection with the diagnostic controller 102 and the stimulation probe 104. If the cable 106 is configured with multiple lead conductors, the cable can include a marked coupling plug to indicate a proper mate with an electrical connector on the stimulation probe 104 or be designed to mate in a particular way with an electrical connector on the stimulation probe. An additional cable 140 connects the ground pad electrode 120 with plug 142 to a ground pad receptacle 144 of the diagnostic controller 102.

The features of diagnostic controller 102 described are for illustration, and diagnostic controllers, such as generators or electrosurgical units suitable for use with stimulation probe 104 may include some, all, or other features than those described below. In one example, the diagnostic controller 102 is capable of operating in monopolar and bipolar modes as well as multiple functions within a mode such as a monopolar stimulation function, a monopolar cutting function, and a monopolar coagulation function. In some examples, a monopolar device can perform a monopolar hemostasis or tissue sealing function. In the monopolar stimulation function, an electrical stimulation signal is provided at a selected amplitude, such as power, and frequency, such as a selected stimulation setting. The stimulation setting can be selected from within a range of suitable stimulation settings having an associated amplitude, such as power, from a range of amplitude or power and frequency from a range of frequencies. For example, electrical energy for a stimulation function may be provided to the active electrode 110 at a relatively low voltage and a continuous current within a range of nominal impedance. In one example, the energy can be applied in the form of bursts of pulses. Each burst typically has a selected duration when the stimulation signal is provided to the active electrode. The pulses can be sinusoidal or square waves and bi-phasic, that is, alternating positive and negative amplitudes.

The diagnostic controller 102 includes a power switch to turn the unit on and off and a setting display to display information regarding the stimulation signal supplied to the stimulation probe 104. The setting display can display an amplitude setting numerically in a selected unit such as watts and the frequency setting numerically in a selected unit such as hertz. The setting display can also include additional information such as pulse forms and ranges of settings.

The diagnostic controller 102 includes a stimulation signal selector comprising amplitude and frequency setting switches that are used to select or adjust the power and frequency settings. A user can push a power setting switch to increase the power setting and push the other power setting switch to decrease the power setting. A user can push a frequency setting switch to increase the frequency setting and push the other frequency setting switch to decrease the frequency setting. The diagnostic controller 102 can also include a set of preprogramed stimulation signal switches to select a stimulation signal from a set of preprogrammed frequency settings. In one embodiment, setting switches are membrane switches, soft keys, or as part of a touchscreen.

In one embodiment, the diagnostic controller 102 provides the stimulation signal to the stimulation probe 104, but the power and frequency levels delivered to the stimulation probe 104 are selectable via controls on the stimulation probe 104 rather than controls on the diagnostic controller 102. In another embodiment, the diagnostic controller 102 is programmed to provide power and frequency levels within a selected range of power and range of frequencies, and the stimulation probe 104 is used to select an output power level and frequency within the preprogrammed range. For instance, the diagnostic controller 102 is programmed to provide a monopolar stimulation signal in a range of power settings and a range of frequencies. The power setting is adjustable within the power range or frequency with the range of frequencies using controls on the stimulation probe 104 or in another location, such as with a foot switch or voice control, rather than with controls on the diagnostic controller 102. Other embodiments of controlling power settings and frequency with controls on the stimulation probe 104 rather than with controls on the diagnostic controller 102 are contemplated.

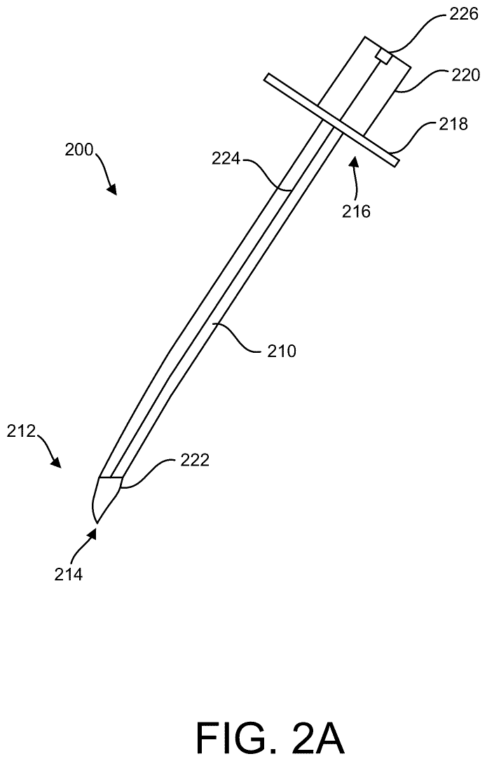

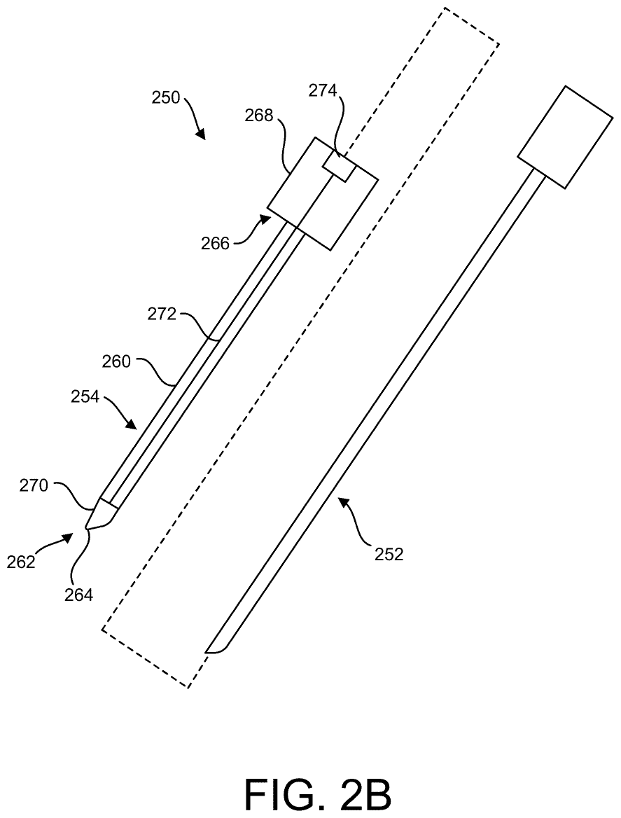

FIGS. 2A and 2B illustrate stimulation probes 200, 250, of many embodiments of stimulation probes, to be used as stimulation probe 104 in the system 100. As illustrated in FIG. 2A, stimulation probe 200 is configured as a Tuohy needle, or an elongated hypodermic needle with a slight curve at the distal end. In the embodiment, the stimulation probe 200 includes an elongated tubular needle 210 having an open, sharp distal end portion 212 with an open and sharp distal tip 214 configured to puncture and advance through tissue toward the pericardium. A proximal end 216 includes wings 218 and a needle hub 220. A distal electrode 222 is disposed on the distal end and operably coupled to an elongated conductor 224 extending along a shaft of the needle to the proximal end 216. The elongated conductor 224 is operably coupled to an electrical connector 226, such as disposed on the needle hub 220. The electrical connector 226 can be coupled to the cable 106 to receive the electrical stimulation signal from the diagnostic controller 102 and deliver the electrical stimulation signal to the distal electrode 222, configured as the active electrode 110. The distal electrode 222 and elongated conductor 224 is formed as conductive arms, wires, traces, other conductive elements, and other electrical pathways formed from electrically conductive material such as metal and may comprise stainless steel, titanium, gold, silver, platinum or any other suitable material. The distal electrode 222 is formed as a conductive element approximately 1 to 2 millimeters in size. The elongated conductor 224 in one example is insulated, or covered with an electrically insulating material.

FIG. 2A illustrates a single distal electrode 222 disposed on the distal tip 214 or near the distal tip 214 of the stimulation probe 200. Other configurations are possible. In one embodiment, multiple electrodes can be included on the stimulation probe, such an electrode on the distal tip and electrodes spaced apart along the elongate portion of the needle. Each electrode can be associated with an insulated elongated conductor and electrically coupled to the electrical connector on the hub.

As illustrated in FIG. 2B, stimulation probe 250 is configured as a microneedle set, or needle-in-needle set, including a micropuncture needle 252 configured to be inserted into a tubular guide needle 254, or Cook needle 254. The guide needle 254 is advanced through the patient 20 toward the pericardium with the micropuncture needle 252 included within. At the pericardium, the micropuncture needle 252 is applied to pierce the fibrous pericardium. Larger bore needles can use greater force than smaller bore needles to penetrate the tough fibrous pericardium, which may propel the needle into contact with the epicardial surface in the presence of respiratory and cardiac motion. A 21 gauge microneedle has fifty-eight percent less surface area than an 18 gauge Tuohy needle and exerts less shearing force. In the embodiment, the guide needle 254 is configured as an elongated tubular needle 260 having an open, sharp distal end portion 262 with an open and sharp distal tip 264 configured to puncture and advance through tissue toward the pericardium. A proximal end 266 includes a needle hub 268. In the example, a distal electrode 270 is disposed on the distal end and operably coupled to an elongated conductor 272 extending along a shaft of the needle to the proximal end 266. The elongated conductor 272 is operably coupled to an electrical connector 274, such as disposed on the needle hub 268. The electrical connector 274 is coupled to the cable 106 to receive the electrical stimulation signal from the diagnostic controller 102 and deliver the electrical stimulation signal to the distal electrode 270, configured as the active electrode 110. The distal electrode 270 and elongated conductor 272 is formed as conductive arms, wires, traces, other conductive elements, and other electrical pathways formed from electrically conductive material such as metal and may comprise stainless steel, titanium, gold, silver, platinum or any other suitable material. The distal electrode 270 is formed as a conductive element approximately 1 to 2 millimeters in size. The elongated conductor 272 in one example is insulated, or covered with an electrically insulating material. In other embodiments, the electrode 270 is disposed on the micropuncture needle 252 or multiple electrodes can be spaced apart along the guide needle 254 or on the micropuncture needle 252. The guide needle 254 is operable as a guide sheath for the micropuncture needle 252. In other embodiments, the stimulation probe 104 is configured as a guide sheath with an active electrode 110 disposed on a distal end or along the sheath.

In some embodiments, the stimulation probes 200, 250 is provided with switches or controller disposed on the hubs 220, 268, respectively to apply the electrical stimulation signal to the electrodes or to adjust or control the electrical stimulation signal applied to the electrodes. For example, the hubs are provided with binary switches to selectively activate or deactivate the electrodes as the electrical stimulation signal is provided to the stimulation probes 200, 250. In other embodiments, the hubs are provided with variable controls, such as variable position or output switches or touch controls to adjust the amplitude or frequency of the electrical stimulation signal or to adjust the amplitude or frequency of the electrical stimulation signal within a range of amplitude or frequency as provided by the diagnostic controller 102.

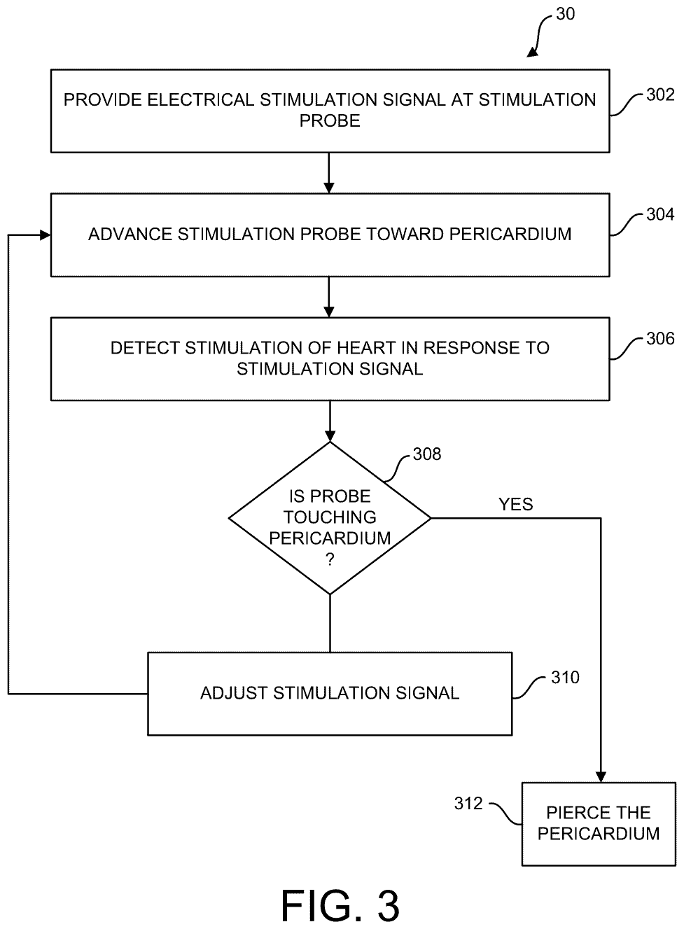

FIG. 3 illustrates a method 300 that can be used with the example system 100. In one embodiment, the method 300 can be used with a system having a puncture device, such as the stimulation probe 104. The puncture device includes a shaft having a proximal portion and a distal portion, the distal portion includes an electrode. In one embodiment, the electrode is configured in monopolar mode. An electrical stimulation signal is provided at the stimulation probe at 302. In one embodiment, a first stimulation signal having a first amplitude and first frequency and having a first stimulation energy is provided to the electrode. The electrode of the puncture device is advanced toward a pericardium of a heart by manipulating the proximal portion of the puncture device at 304. Stimulation of the heart is detected in response to the stimulation signal at 306. In one embodiment, a first stimulation of the heart in response to the first stimulation signal. If the probe is not in mechanical contact with the heart, such as abutting the heart, at 308, the stimulation signal is adjusted at 310. In one embodiment, a second stimulation signal having a second stimulation signal having a second amplitude and second frequency and having a second stimulation energy is provided to the electrode. The signal is adjusted such that the first stimulation energy is greater than the second stimulation energy. For instance, the first stimulation signal includes a greater amplitude than the second stimulation signal. The stimulation probe provides the adjusted, or second electrical stimulation signal. The stimulation energy used to stimulate the heart becomes less as the probe is advanced towards the heart again at 304. If the process of reducing the stimulation energy upon stimulating the heart is repeated enough, the heart will be stimulated by a small amount of stimulation energy that is applied as the probe is adjacent to the pericardium. Once a determination is made that the probe is in contact with the pericardium at 308, the pericardium can be pierced with the probe at 312. In some embodiments, the pericardium can be mechanically pierced with a needle or sharp tip. In other embodiments, puncture device is a radiofrequency (RF) crossing device. For instance, the probe can include an electrode to apply an RF signal supplied by a generator to penetrate the pericardium. In this embodiment, the RF signal is much greater than a stimulation signal applied to the probe.

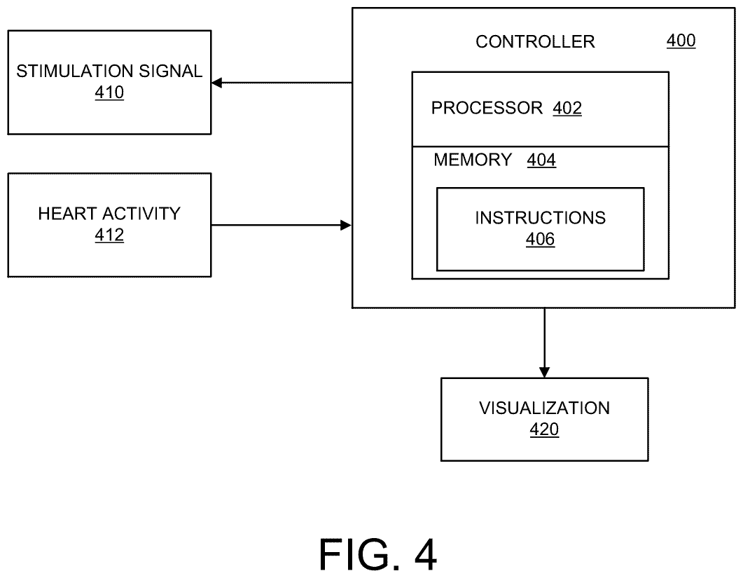

FIG. 4 illustrates an embodiment of a controller 400 that can be used with the system 100, which controller 400 includes features of the diagnostic controller 102 and the recording controller 112. The controller 400 can be implemented to provide the electrical stimulation signal of the selected amplitude and frequency to a stimulation probe, detect a stimulation of the heart in response to the electrical stimulation signal, and adjust at least one of the selected amplitude and frequency of the electrical stimulation signal. The controller 400 can include a processor 402 and a memory 404. The memory 404 stores processor executable instructions 406. In one embodiment, the processor executable instructions 406 can be in the form of a program, such as a computer program or application. The processor 402 can execute the instructions 406 that can be included in configuring the controller 400. In one example, the controller 400 can be implemented as a generator or specialty medical device or to include a general-purpose computing device such as a laptop computer, a workstation, a desktop computer, a tablet, or a smartphone. The controller 400 can include additional components such as a display, a touchscreen, speakers or other output devices, buttons, switches, other controls, a keyboard or other input devices, or communication circuitry such as computer network adapters. The controller 400 may be implemented in a variety of architectures and components, such as the processor 402 and memory 404, may be distributed in various locations.

In one embodiment, the processor 402 includes a plurality of main processing cores to run an operating system and perform general-purpose tasks on an integrated circuit. The processor 402 may also include built-in logic or a programmable functional unit, also on the same integrated circuit. In additional to multiple general-purpose, main processing cores and the application processing unit, controller 400 can include other devices or circuits such as graphics processing units or neural network processing units.

Memory 404 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 402. Any such computer storage media may be part of the controller 400 and implemented as memory 404. Memory 404 is a non-transitory, processor readable memory device. Accordingly, a propagating signal by itself does not qualify as storage media or memory 404.

The controller 400 is configured to provide outputs and receive inputs or information from the system. For example, the controller 400 provides a selected stimulation signal 410 based on a selected or controller-determined amplitude and frequency of an electrical signal either continuously or at a selected time. The controller 400 includes or is connected to electrical circuitry to generate the selected stimulation circuitry and provide it to a receptacle, such as receptacle 134 and ground pad receptacle 144. The controller 400 is also configured to receive heart activity information 412 that can serve in a determination as to whether the heart has been stimulated in response to the stimulation signal. For example, the heart activity information 412 can be received in the form of electrode signals from an ECG interface, transducer signals from an echocardiogram interface, signals representative of an X-ray such as from a C-arm, a heart map, or other information. Other inputs can include parameters regarding the stimulation probe, number of electrodes on the stimulation probe, and patient information. The controller 400 can be configured to generate a visualization 420 that can be based on the selected stimulation signal 410 or the heart activity information 412, the determination of whether the heart has been stimulated, and other features. In some examples, the visualization 420 can correspond with other information such as audio alarms or other sounds, such as an alarm if the controller detects the heart has been stimulated and another alarm if the controller determines the stimulation probe is touching the pericardium.

In one embodiment, the instruction 406 can include instructions to provide an electrical stimulation signal of the selected amplitude and frequency to a stimulation probe, instructions to detect a stimulation of the heart in response to the electrical stimulation signal, and instructions to adjust at least one of the selected amplitude and frequency of the electrical stimulation signal. Additional instructions can include instructions to receive inputs and instructions to generate visualization.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method of epicardial access of a heart, the method comprising:
providing a puncture device including a shaft having a proximal portion and a distal portion, the distal portion including an electrode;
providing a first stimulation signal having a first amplitude and frequency having a first stimulation energy to the electrode;
advancing the electrode of the puncture device toward a pericardium of the heart by manipulating the proximal portion of the puncture device;
detecting a first stimulation of the heart in response to the first stimulation signal;
providing a second stimulation signal having a second stimulation signal having a second amplitude and frequency having a second stimulation energy to the electrode; and
detecting a second stimulation of the heart in response to the second stimulation signal, based on the electrode contacting the epicardium of the heart;
wherein the first stimulation energy is greater than the second stimulation energy.

2. The method of claim 1, wherein upon detecting a stimulation of the heart in response to the electrical stimulation signal, determining whether the stimulation probe is touching the pericardium.

3. The method of claim 2, wherein if the stimulation probe is touching the pericardium, piercing the pericardium.

4. The method of claim 2, wherein if the stimulation probe is not touching the pericardium, further advancing the stimulation probe toward the pericardium, the stimulation probe providing the adjusted at least one of the amplitude and frequency of the electrical stimulation signal.

5. The method of claim 1, wherein the adjusting at least one of the selected amplitude and frequency includes lowering the amplitude.

6. The method of claim 1, wherein the stimulation probe includes a needle configured to pierce the pericardium.

7. The method of claim 6, wherein the needle is a Tuohy needle.

8. The method of claim 6, wherein the needle is included in a micropuncture needle set.

9. The method of claim 1, wherein the stimulation probe includes a delivery sheath.

10. The method of claim 9, wherein the delivery sheath includes an electrode.

11. The method of claim 1, wherein the stimulation probe includes an electrode configured in a monopolar mode.

12. The method of claim 1, wherein the detecting the stimulation includes detecting the stimulation via a QRS wave of the heart.

13. The method of claim 1, wherein the detecting the stimulation includes detecting the stimulation via determining changes in an electrogram signal.

14. The method of claim 1, wherein the puncture device is a radio-frequency (RF) crossing device.

15. A system to access an epicardium of a heart, the system comprising:
a stimulation probe configured to advance toward a pericardium and configured to provide an electrical stimulation signal of a selected amplitude and frequency; and
a controller coupled to the to the stimulation probe, the controller configured to provide the electrical stimulation signal of the selected amplitude and frequency to the stimulation probe and adjust at least one of the selected amplitude and frequency of the electrical stimulation signal based on a detected stimulation of the heart in response to the electrical stimulation signal.

16. The system of claim 15, wherein the adjusting at least one of the selected amplitude and frequency includes lowering the amplitude.

17. A system to access an epicardium of a heart, the system comprising:
a puncture device including a shaft having a proximal portion and a distal portion, the distal portion including an electrode, the puncture device configured to advance toward a pericardium of the heart by manipulating the proximal portion of the puncture device; and
a controller coupled to the puncture device, the controller configured to provide a first stimulation signal having a first amplitude and frequency having a first stimulation energy to the electrode, detect a first stimulation of the heart in response to the first stimulation signal, provide a second stimulation signal having a second stimulation signal having a second amplitude and frequency having a second stimulation energy to the electrode, and detect a second stimulation of the heart in response to the second stimulation signal, based on the electrode contacting the epicardium of the heart;
wherein the first stimulation energy is greater than the second stimulation energy.

18. The system of claim 17, wherein the puncture device is one of a radio-frequency (RF) crossing device and a needle configured to pierce the pericardium.

19. The system of claim 18, wherein the needle is one of a Tuohy needle and a micropuncture needle set.

20. The system of claim 17, wherein the controller is further configured to generate a visualization on a display.
